(19) United States
(12) Plant Patent Application Publication
(10) Pub. No.: US 2002/0184679 P1
(43) Pub. Date: Dec. 5, 2002

(54) Chrysanthemum plant named 'Gedi One Ter'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,962

(22) Filed: Jun. 4, 2001

(51) Int. Cl. A01H 5/00

(52) U.S. Cl. PLT/288

(57) ABSTRACT

A new and distinct Chrysanthemum plant cultivar characterized by large decorative type inflorescences, consistent flowering response to short days, blooming consistently after 49 days of short day length, free branching habit, and a consistent natural season flowering habit during the last two weeks of August.

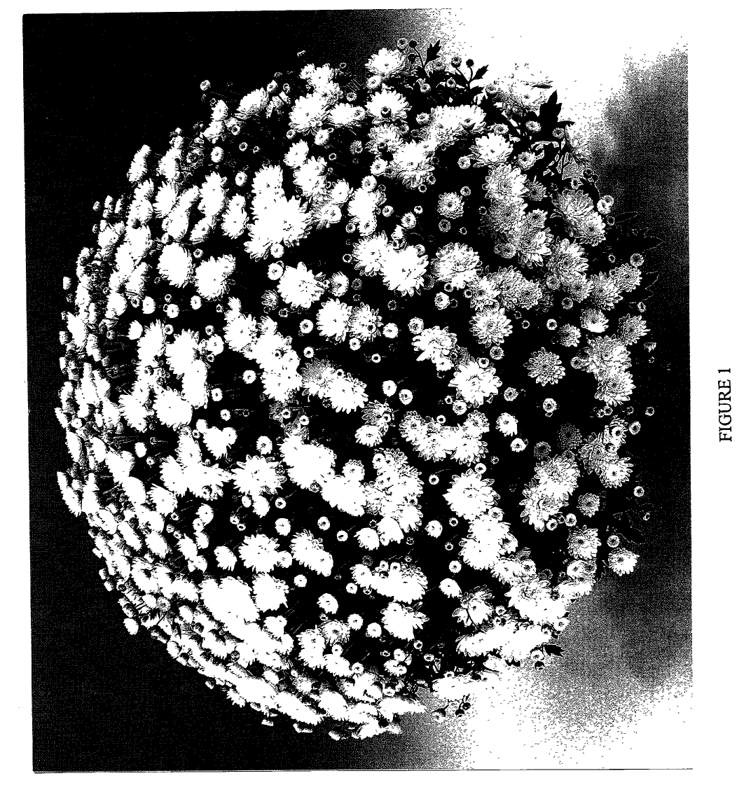

### BACKGROUND OF THE INVENTION

The present invention relates to a new and distinct cultivar of Chrysanthemum plant, botanically known as Dendranthema morifolium and herein after referred to by the cultivar name ‘GEDI ONE TER.’

The new cultivar is a product of hybridization of a female parent Chrysanthemum cultivar (unpatented) and a male parent ‘Subito,’ (unpatented.) The new cultivar was discovered and selected by the inventor in October 1997.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE TER’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE TER.’ These characteristics in combination distinguish ‘GEDI ONE TER’ as a new and distinct Chrysanthemum cultivar:

1. Large decorative type inflorescence;

2. Consistent flowering response to short days, blooming consistently after 49 days of short day length;

3. Free branching habit; and

4. Consistent natural season flowering habit during the last two weeks of August.

Plants of the new cultivar are similar to plants of the female and male parent cultivars in most horticultural characteristics, however plants of the new cultivar differed from plants of the parent cultivars in several characteristics. In comparison the new cultivar differed from the parents in the following characteristics:

1. The new cultivar has a wider diameter flower than the female parent;

2. The new cultivar has many more ray florets than the female parent;

3. The new cultivar naturally blooms three weeks earlier than the female parent;

4. The new cultivar has a wider diameter flower than the male parent;

5. The new cultivar has many more ray florets than the male parent; and

6. The new cultivar has different color ray florets than the male parent.

### BRIEF DESCRIPTION OF THE PHOTOGRAPHS

The photograph shows a plant of the new cultivar which was grown in a 2-gallon container. A single cutting was grown in the container, and the plant was grown in outdoor field conditions.

### DETAILED BOTANICAL DESCRIPTION

In the following description, color references are made to The Royal Horticultural Society Colour Chart except where general terms of ordinary dictionary significance are used. The following observations and measurements describe plants grown in a commercial field in Oxnard, Calif. in the month of September 2000. The growing conditions range from 60° to 65° F. at night to 70° to 85° F. during the day. Measurements and numerical values represent averages of typical flowering types.

Botanical classification: Dendranthema morifolium cultivar ‘GEDI ONE TER’

Commercial classification: Garden-type Chrysanthemum

Parentage: Hybridization of female parent Chrysanthemum plant (unpatented) and male parent Chrysanthemum cultivar ‘Subito’

### Propagation

Time to Rooting: 7 to 14 days at approximately 21° C.

Rooting habit: Fine, fibrous

### Plant

Growth Habit: Mounding herbaceous perennial

Height: Approximately 25 cm

Growth rate: Moderate

Branching Characteristics: Free Branching

### Foliage

Leaf:

Arrangement.—Alternate single

Average length.—Approximately 7.8 cm

Average width.—Approximately 4 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color.—young foliage upper side: Near 137B young foliage under side: Near 137B mature foliage upper side: Near 137A mature foliage under side: Near 137A venation upper side: Near 138C venation underside: Near 138C

Petiole:

Average length.—Approximately 2.5 cm

### Bloom

Inflorescence:

Flowering habit.—Induced by darkness period greater than 13.5 hours, approximately 49 days of appropriate day length required to induce and develop blooms

Inflorescence form.—Decorative

Natural flowering season.—Approximately the last two weeks in August

Number of in florescences per stem.—Approximately 13

Inflorescence diameter.—Approximately 7 cm

Inflorescence depth.—2.0 cm

Inflorescence longevity on plant.—Approximately 26-29 days

Persistence.—Persistent

Ray Florets:

Appearance.—Matte

Texture.—Smooth

Average number per flower.—300

Shape.—Oblanceolate

Margin.—Entire

Apex.—Obtuse

Length.—Approximately 4 cm

Width.—Approximately 1 cm

Color.—upper surface at first opening: Near 158B upper surface at maturity: Near 155D upper surface at fading: Near 155D under surface at first opening: Near 158C under surface at maturity: Near 155D under surface at fading: Near 155D

Disc florets:

Appearance.—Shiny

Texture.—Smooth

Average number per flower.—Approximately 40

Shape.—Cylindric

Apex.—Obtuse

Average length.—Approximately 1 cm

Average width.—Approximately 0.2 cm

Color.—at first opening: Near 8C at maturity: Near 12B at fading: Near 15C

Peduncle:

Length.—at terminal end (shortest): Approximately 7 cm at lateral end (longest): Approximately 12 cm

Strength.—Moderate

Color.—Near 137C

Habit.—Erect

Inflorescence bud:

Length.—Approximately 1 cm

Diameter.—Approximately 1 cm

Form.—Globular

Color.—Near 8D

Sepals:

Appearance.—Matte

Texture.—Lightly pubescent

Number.—Approximately 40 to 50

Shape.—Ovate

Margin.—Entire

Apex.—Acute

Color.—upper side: Near 137A under side: Near 137A

### Reproductive Organs

Ray Florets:

Pistillate.—Number of pistils per flower: 1 Stigma shape: 2 branched Stigma color: Near 16D Style color: Near 16D Style length: 0.6 cm

Disc Florets:

Pistillate.—Number ofpistils per flower: 1 Stigma shape: Cylindric Style length: 0.5 cm

Staminate.—Number of stamens per flower: Approximately 5 Anther shape: Tubular Anther color: Near 16A Pollen color: Near 17B

What is claimed is:

1. A new and distinct cultivar of Chrysanthemum plant named ‘GEDI ONE TER’ as herein illustrated and described.
